(19) United States
(12) Patent Application Publication
(10) Pub. No.: US 2026/0366083 A1
(43) Pub. Date: Nov. 19, 2026

(54) SYSTEMS AND METHODS FOR LONGITUDINAL MRD MONITORING

(71) Applicant: Predicine, Inc., Hayward, CA (US)

(72) Inventors: Kemin ZHOU, Hayward, CA (US); Pan DU, Hayward, CA (US); Shidong JIA, Hayward, CA (US)

(21) Appl. No.: 19/722,034

(22) Filed: Jun. 26, 2026

Related U.S. Application Data
(63) Continuation of application No. PCT/US2025/046519, filed on Sep. 16, 2025.
(60) Provisional application No. 63/695,635, filed on Sep. 17, 2024.
(60) Provisional application No. 63/779,743, filed on Mar. 28, 2025.

(51) Int. Cl. C12Q 1/6886

(52) U.S. Cl. CPC C12Q 1/6886; C12Q 2600/156; C12Q 2600/158; C12Q 2600/16; G01N 2800/54

(57) ABSTRACT

Described herein are systems and methods for longitudinal minimal residual disease monitoring. Biomarkers may be detected at various time points to generate data indicative of the presence or absence of minimal residual disease (MRD) in a subject. The biomarkers may be detected at various time points during treatment. The biomarkers may also be detected after treatments and may be used to predict treatment resistance of disease such as cancer.

### CROSS-REFERENCE

This application is a continuation of International Application No. PCT/US2025/046519, filed Sep. 16, 2025, which claims the benefit of U.S. Provisional Application No. 63/695,635 filed Sep. 17, 2024, and U.S. Provisional Application No. 63/779,743, filed Mar. 28, 2025, each is incorporated by reference herein in its entirety.

### BACKGROUND

Biological samples such as cell-free bodily fluids may be used to diagnose diseases such as cancer. Biological samples may be detected to create a panel. A panel may be a collection of unique molecules or differentially expressed markers or variants. A panel may allow for a comparison during testing of unknown molecules. A biological sample of a patient may be collected to compare against the panel. Mutations may be detected in the biological sample by using sequencing methods. Diagnostics using panels may be used to detect disease states such as minimum residual disease.

### SUMMARY

Provided herein are systems and methods for detection of the presence or absence of cancer in a subject. The systems and methods provided herein may use panels to detect disease states such as minimum residual disease in a subject.

Disclosed herein in some embodiments are methods, systems, and devices for monitoring disease state and predicting treatment resistance monitoring.

Disclosed herein in one embodiment is a method for identifying a presence or an absence of minimal residual disease (MRD) in a subject, comprising: (a) assaying nucleic acid molecules from a first biological sample obtained or derived from the subject; (b) detecting one or more biomarkers from the nucleic acid molecules from the first biological sample based at least in part on the assaying of (a); (c) generating a plurality of probe nucleic acids that are customized for the subject, wherein the plurality of probe nucleic acids comprise sequences corresponding a set of biomarkers, wherein the set of biomarkers comprise (i) at least a subset of the one or more biomarkers and (ii) one or more previously identified biomarkers; (d) using the plurality of probe nucleic acids, sequencing cell free nucleic acids from a second biological sample obtained or derived from the subject to detect the presence or absence of a subset of the set of biomarkers; (e) computer processing the subset of the set of biomarkers to predict the presence of minimal residual disease (MRD) in the subject.

In some embodiments, generating the plurality of probe nucleic acid comprises modifying a pre-existing plurality of probe nucleic acids by adding or removing one or more probes. In some embodiments, the generating comprises performing a comparison the one or more biomarkers with a set of previously identified biomarkers. In some embodiments, the one or more biomarkers or the previously identified biomarkers comprise tumor-associated alterations selected from the group consisting of: single nucleotide variants (SNVs), insertions or deletions (indels), and rearrangements. In some embodiments, the one or more biomarkers or the previously identified biomarkers comprise differentially expressed markers or variants. In some embodiments, the previously identified biomarkers were identified prior to the administration of a treatment regimen to the subject. In some embodiments, the previously identified biomarkers were identified during the administration of a treatment regimen of the subject. In some embodiments, the previously identified biomarkers comprise biomarkers that were identified at plurality of different time points.

In some embodiments, the plurality of different time points comprises at least two time points during the administration of a treatment regimen of the subject. In some embodiments, the method comprises detecting the presence or absence of minimal residual disease in the subject at an accuracy of at least about 60%, at least about 70%, at least about 75%, at least about 80%, at least about 85%, at least about 90%, at least about 95%, at least about 98%, or at least about 99%. In some embodiments, the method comprises detecting the presence or absence of minimal residual disease in the subject in the subject at a negative predictive value of at least about 60%, at least about 70%, at least about 75%, at least about 80%, at least about 85%, at least about 90%, at least about 95%, at least about 98%, or at least about 99%. In some embodiments, the method comprises detecting the presence or absence of minimal residual disease in the subject at a sensitivity of at least about 60%, at least about 70%, at least about 75%, at least about 80%, at least about 85%, at least about 90%, at least about 95%, at least about 98%, or at least about 99%. In some embodiments, the method comprises detecting the presence or absence of minimal residual disease in the subject at a specificity of at least about 60%, at least about 70%, at least about 75%, at least about 80%, at least about 85%, at least about 90%, at least about 95%, at least about 98%, or at least about 99%. In some embodiments, the method comprises detecting the presence or absence of minimal residual disease in the subject at a positive predictive value of at least about 60%, at least about 70%, at least about 75%, at least about 80%, at least about 85%, at least about 90%, at least about 95%, at least about 98%, or at least about 99%.

In some embodiments, the first biological sample is selected from the group consisting of: a cell-free deoxyribonucleic acid (cfDNA) sample, a cell-free ribonucleic acid (cfRNA) sample, a plasma sample, a serum sample, a buffy coat sample, a peripheral blood mononuclear cell (PBMC) sample, a red blood cell sample, a urine sample, a urine cell pellet sample, a saliva sample, tissue biopsy, pleural fluid sample, peritoneal fluid sample, amniotic fluid sample, cerebrospinal fluid sample, lymphatic fluid sample, sweat sample, tear sample, semen sample, or any derivative thereof, and any combination thereof. In some embodiments, the first biological sample may comprise a plasma sample. In some embodiments, the first biological sample may comprise a urine sample. In some embodiments, the first biological sample may comprise a tumor tissue sample. In some embodiments, the second biological sample may comprise a urine sample. In some embodiments, the second biological sample may comprise a urine cell pellet sample. In some embodiments, the second biological sample may comprise a blood sample. In some embodiments, the nucleic acid molecules of the first biological sample may comprise DNA molecules.

In some embodiments, the DNA molecules comprise cell-free DNA (cfDNA) molecules. In some embodiments, the nucleic acid molecules of the first biological sample may comprise RNA molecules. In some embodiments, the RNA molecules comprise cell-free RNA (cfRNA) molecules.

In another embodiment, disclosed herein is a method for predicting presence or an absence of treatment-resistant minimal residual disease (MRD) in a subject, comprising: (a) assaying nucleic acid molecules from a first biological sample obtained or derived from the subject at a first time point; (b) detecting a first set of biomarkers from the DNA molecules from the first biological sample based at least in part on the assaying of (a); (c) assaying nucleic acid molecules from a second biological sample obtained or derived from the subject at a second time point; (d) detecting a second set of biomarkers from the nucleic acid molecules from the second biological sample based at least in part on the assaying of (c); (e) generating a plurality of probe nucleic acids that are customized for the subject, wherein the probe nucleic acids comprise (i) sequences corresponding to at least a subset of the first set of biomarkers and (ii) sequences corresponding to at least a subset of the second set of biomarkers; (f) using the plurality of probe nucleic acids, sequencing cell free nucleic acids (cfDNA) from a third biological sample obtained or derived from the subject at a third time point to detect the presence or absence of the subset of the pooled set of biomarkers; (g) processing the subset of the pooled set of biomarkers to detect the presence of treatment-resistant minimal residual disease (MRD) in the subject.

In some embodiments, the first or second biological sample is selected from the group consisting of: a cell-free deoxyribonucleic acid (cfDNA) sample, a cell-free ribonucleic acid (cfRNA) sample, a plasma sample, a serum sample, a buffy coat sample, a peripheral blood mononuclear cell (PBMC) sample, a red blood cell sample, a urine sample, a urine cell pellet sample, a saliva sample, tissue biopsy, pleural fluid sample, peritoneal fluid sample, amniotic fluid sample, cerebrospinal fluid sample, lymphatic fluid sample, sweat sample, tear sample, semen sample, or any derivative thereof, and any combination thereof. In some embodiments, the first or second biological sample may comprise a plasma sample. In some embodiments, the first or second biological sample may comprise a urine sample. In some embodiments, the first or second biological sample may comprise a tumor tissue sample. In some embodiments, the first or second biological sample is obtained or derived from the subject using an ethylenediaminetetraacetic acid (EDTA) collection tube, a cell-free RNA collection tube, or a cell-free deoxyribonucleic acid (DNA) collection tube, other blood collection tube, and CTC collection tubes.

In some embodiments, the third biological sample is selected from the group consisting of: a cell-free deoxyribonucleic acid (cfDNA) sample, a cell-free ribonucleic acid (cfRNA) sample, a plasma sample, a serum sample, a buffy coat sample, a peripheral blood mononuclear cell (PBMC) sample, a red blood cell sample, a urine sample, a urine cell pellet sample, a saliva sample, tissue biopsy, pleural fluid sample, peritoneal fluid sample, amniotic fluid sample, cerebrospinal fluid sample, lymphatic fluid sample, sweat sample, tear sample, semen sample, or any derivative thereof, and any combination thereof. In some embodiments, the third biological sample may comprise the plasma sample. In some embodiments, the third biological sample may comprise the urine sample. In some embodiments, the third biological sample may comprise a urine cell pellet sample. In some embodiments, the third biological sample may comprise a blood sample. In some embodiments, the third biological sample is obtained or derived from the subject using an ethylenediaminetetraacetic acid (EDTA) collection tube, a cell-free RNA collection tube, or a cell-free deoxyribonucleic acid (DNA) collection tube, other blood collection tube, and CTC collection tubes.

In some embodiments, the method comprises detecting the presence or absence of treatment-resistant minimal residual disease in the subject at an accuracy of at least about 60%, at least about 70%, at least about 75%, at least about 80%, at least about 85%, at least about 90%, at least about 95%, at least about 98%, or at least about 99%. In some embodiments, the method comprises detecting the presence or absence of treatment-resistant minimal residual disease in the subject at a positive predictive value of at least about 60%, at least about 70%, at least about 75%, at least about 80%, at least about 85%, at least about 90%, at least about 95%, at least about 98%, or at least about 99%. In some embodiments, the method comprises detecting the presence or absence of treatment-resistant minimal residual disease in the subject in the subject at a negative predictive value of at least about 60%, at least about 70%, at least about 75%, at least about 80%, at least about 85%, at least about 90%, at least about 95%, at least about 98%, or at least about 99%. In some embodiments, the method comprises detecting the presence or absence of treatment-resistant minimal residual disease in the subject at a sensitivity of at least about 60%, at least about 70%, at least about 75%, at least about 80%, at least about 85%, at least about 90%, at least about 95%, at least about 98%, or at least about 99%. In some embodiments, the method comprises detecting the presence or absence of treatment-resistant minimal residual disease in the subject at a specificity of at least about 60%, at least about 70%, at least about 75%, at least about 80%, at least about 85%, at least about 90%, at least about 95%, at least about 98%, or at least about 99%.

In some embodiments, the third biological sample is obtained or derived from the subject using an ethylenediaminetetraacetic acid (EDTA) collection tube, a cell-free RNA collection tube, or a cell-free deoxyribonucleic acid (DNA) collection tube, other blood collection tube, and CTC collection tubes. In some embodiments, the nucleic acid molecules of the first or second biological sample may comprise DNA molecules. In some embodiments, the DNA molecules comprise cell-free DNA (cfDNA) molecules. In some embodiments, the nucleic acid molecules of the first or second biological sample may comprise RNA molecules. In some embodiments, the RNA molecules comprise cell-free RNA (cfRNA) molecules. In some embodiments, the cell free nucleic acids comprise cfDNA. In some embodiments, the cell free nucleic acids comprise cfRNA.

In some embodiments, (a) comprises subjecting the first biological sample to conditions that are sufficient to isolate, enrich, or extract the DNA molecules. In some embodiments, the method further comprises fractionating the first biological sample of the subject to obtain the DNA molecules, wherein the first biological sample is a whole blood sample. In some embodiments, at least one of the nucleic acid molecules are assayed using DNA sequencing to produce nucleic acid sequencing reads. In some embodiments, the DNA sequencing comprises whole exome sequencing. In some embodiments, the method further comprises filtering at least a subset of the nucleic acid sequencing reads based on a quality score.

In some embodiments, the method further comprises performing error correction on the nucleic acid sequencing reads using sample barcodes or molecular barcodes attached to at least one of the DNA molecules. In some embodiments, the method further comprises performing at least one of single-stranded consensus calling and double-stranded consensus calling on the nucleic acid sequencing reads, thereby suppressing sequencing and PCR errors in the nucleic acid sequencing reads. In some embodiments, the sequencing is performed at a depth of at least 100×. In some embodiments, the sequencing is performed at a depth of at least 1,000×. In some embodiments, the sequencing is performed at a depth of at least 10,000×. In some embodiments, the sequencing is performed at a depth of at least 100,000×. In some embodiments, the sequencing comprises sequencing nucleic acids derived from the first biological sample.

In some embodiments, the sequencing comprises sequencing nucleic acids derived from the second biological sample. In some embodiments, the assaying comprises nucleic acid amplification. In some embodiments, the sequencing comprises nucleic acid amplification. In some embodiments, the nucleic acid amplification comprises polymerase chain reaction (PCR) or isothermal amplification.

In some embodiments, the cancer is selected from the group consisting of: genitourinary cancer, prostate cancer, bladder cancer, and any combination thereof. In some embodiments, the cancer comprises the bladder cancer. In some embodiments, the bladder cancer is a muscle invasive bladder cancer. In some embodiments, the subject is asymptomatic for the cancer. In some embodiments, the first biological sample is obtained or derived from the subject prior to the subject receiving a therapy for the cancer. In some embodiments, the first biological sample or the second biological sample is obtained or derived from the subject during a therapy for the cancer. In some embodiments, the second biological sample is obtained or derived from the subject after receiving a therapy for the cancer. In some embodiments, the therapy is selected from the group consisting of: surgical resection, chemotherapy, radiotherapy, immunotherapy, cell therapy, adjuvant therapy, neoadjuvant therapy, androgen deprivation therapy, and a combination thereof.

In some embodiments, the first biological sample is obtained or derived from the subject via a transurethral resection of bladder tumor. In some embodiments, the first biological sample is obtained or derived from the subject after performing a transurethral resection of bladder tumor. In some embodiments, the method further comprises identifying a clinical intervention for the subject based at least in part on the detected presence or the absence of the cancer. In some embodiments, the clinical intervention is selected from a plurality of clinical interventions. In some embodiments, the clinical intervention is selected from the group consisting of: surgical resection, chemotherapy, radiotherapy, immunotherapy, adjuvant therapy, neoadjuvant therapy, androgen deprivation therapy, and a combination thereof. In some embodiments, the surgical resection is a transurethral resection of bladder tumor (TURBT) or a repeat transurethral resection of bladder tumor.

In some embodiments, the method further comprises administering the clinical intervention to the subject. In some embodiments, the plurality of probes comprise nucleic acid primers. In some embodiments, the plurality of probes comprise nucleic acid capture probes.

In some embodiments, the plurality of probes have sequence complementarity with at least a portion of nucleic acid sequences of the set of biomarkers. In some embodiments, the plurality of probes comprise at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 different probes. In some embodiments, sequencing comprises using a fixed plurality of probes wherein the probes of the fixed plurality of probes comprises probes that do not comprise sequences of the subset of the set of biomarkers.

In some embodiments, the method further comprises determining a likelihood of the determination of the presence or the absence of the cancer in the subject. In some embodiments, the method further comprises monitoring the presence or the absence of the cancer in the subject, wherein the monitoring comprises assessing the presence or the absence of the cancer in the subject at each of a plurality of time points.

In some embodiments, a difference in the assessment of the presence or the absence of the cancer in the subject among the plurality of time points is indicative of one or more clinical indications selected from the group consisting of: (i) a diagnosis of the cancer, (ii) a prognosis of the cancer, and (iii) an efficacy or non-efficacy of a course of treatment for treating the cancer of the subject. In some embodiments, the prognosis comprises an expected progression-free survival (PFS) or overall survival (OS). In some embodiments, the method further comprises determining, among the set of biomarkers, a mutant allele frequency of a set of somatic mutations.

In some embodiments, the method further comprises determining a circulating tumor DNA (ctDNA) fraction of the cancer of the subject based at least in part on the set of mutant allele frequencies. In some embodiments, the method further comprises determining a tumor mutational burden (TMB) of the cancer of the subject.

In some embodiments, the method further comprises determining an abnormality score of the cancer of the subject based at least in part on the set of mutant allele frequencies. In some embodiments, the method further comprises applying a treatment based on the detection of a minimum residual disease or a treatment-resistant minimum residual disease. In some embodiments, the method further comprises modifying a treatment based on the detection of the minimum residual disease or the treatment-resistant minimum residual disease.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A illustrates non-limiting examples of tissue types that may be analyzed using the method.

FIG. 1B illustrates a non-limiting example of a method for longitudinal MRD monitoring sampling.

FIG. 2 illustrates a non-limiting example of exemplary MRD monitoring and resistance monitoring bi-directional data.

FIG. 3A illustrates a non-limiting example of data illustrating mutation-level bi-directional allele frequency values for a Patient 1.

FIG. 3B illustrates another non-limiting example of data illustrating mutation-level bi-directional allele frequency values for a Patient 9.

FIG. 4 illustrates a non-limiting example of data illustrating a cfDNA MRD assay for various mutations.

FIG. 5 shows a computer system that is programmed or otherwise configured to implement methods provided herein.

FIG. 6 illustrates an exemplary workflow for longitudinal MRD monitoring in both DNA and RNA by assaying with a sufficiently identical probe panel for both DNA and RNA.

FIG. 7 illustrates an exemplary workflow for longitudinal MRD monitoring utilizing structural variant (SV) detection with personalized probe panels.

FIG. 8A shows a non-limiting example of a deletion subtype of a SV.

FIG. 8B shows a non-limiting example of an insertion subtype of a SV.

FIG. 8C shows a non-limiting example of a duplication subtype of a SV.

FIG. 8D shows a non-limiting example of an inversion subtype of a SV.

FIG. 8E shows a non-limiting example of a translocation subtype of a SV.

FIG. 9 illustrates an exemplary workflow for longitudinal MRD monitoring utilizing methylation patterns, SVs, and genetic mutations.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### TERMS AND DEFINITIONS

As used herein, the singular forms “a,” “an,” and “the” include plural references unless the context clearly dictates otherwise. Any reference to “or” herein is intended to encompass “and/or” unless otherwise stated.
As used herein, the phrases “at least one,” “one or more,” and “and/or” are open-ended expressions that are both conjunctive and disjunctive in operation. For example, each of the expressions “at least one of A, B and C,” “at least one of A, B, or C,” “one or more of A, B, and C”, “one or more of A, B, or C” and “A, B, and/or C” means A alone, B alone, C alone, A and B together, A and C together, B and C together, or A, B and C together. As used herein, the phrase “at most three” can mean less than one, one, two, or three.
Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.
The terms “subject,” “individual,” and “patient” may be used interchangeably and refer to humans, as well as non-human mammals (e.g., non-human primates, canines, equines, felines, porcines, bovines, ungulates, lagomorphs, rodents, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker. In some embodiments, the subject may be under the care of a dental professional.
As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a therapeutic to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder.

Disclosed herein, some embodiments may comprise methods, systems, and devices for monitoring disease state and predicting treatment resistance monitoring.

The present disclosure provides multiple improvements relating to diagnostic panels. For example, in some cases, conventional use of diagnostics panels may require a normal control. However, a normal control may be inaccurate as mutations accrue and change over time. As such, panels may not be suitable for monitoring over time because the baseline “normal” collection of mutations may changes over time, for example during a treatment. Panels may not be sufficient to detect biomarkers that predict resistance to treatment for a subject. The present disclosure provides methods and systems that allow the generation of panels that take in to account the changes in mutations or genetic aberrations of a subject over time. For example, the methods and systems allow for modifications to a panel over time or changes to a reference normal in a panel over time which may facilitate treatment resistance monitoring and bi-directional minimum residual disease monitoring.

Disclosed herein in one embodiment may be a method for identifying a presence or an absence of minimal residual disease (MRD) in a subject. In some embodiments, the method may comprise assaying nucleic acid molecules from a first biological sample. In some embodiments, the first biological sample may be obtained or derived from the subject. In some embodiments, the method may further comprise detecting one or more biomarkers from the nucleic acid molecules from the first biological sample based at least in part on the assaying. In some embodiments, the method may comprise assaying about two, three, four, five, six, seven, eight, nine, ten, twenty, thirty, forty, fifty, sixty, seventy, eighty, ninety, one hundred, or more than about one hundred molecules from the first biological example. In some embodiments, the method may further comprise generating a plurality of probe nucleic acids. In some embodiments, the plurality of probe nucleic acids may be customized for the subject. In some embodiments, the plurality of probe nucleic acids may comprise sequences corresponding a set of biomarkers. In some embodiments, the set of biomarkers may comprise at least a subset of the one or more biomarkers. In some embodiments, the set of biomarkers may comprise one or more previously identified biomarkers. In some embodiments, the set of biomarkers may comprise about about two, three, four, five, six, seven, eight, nine, ten, twenty, thirty, forty, fifty, sixty, seventy, eighty, ninety, one hundred, or more than about one hundred previously identified biomarkers. In some embodiments, the method may further comprise using the plurality of probe nucleic acids to sequence cell free nucleic acids. In some embodiments, the cell free nucleic acids may be obtained from a second biological sample. In some embodiments, the second biological sample may be obtained or derived from the subject. In some embodiments, the method may further comprise detecting a presence of a subset of the set of biomarkers. In some embodiments, the method may further comprise detecting an absence of a subset of the set of biomarkers. In some embodiments, the method may further comprise computer processing the subset of the set of biomarkers. In some embodiments, the method may further comprise executing a computer process to predict the presence of minimal residual disease (MRD) in the subject.

In some embodiments, generating the plurality of probe nucleic acids may comprise modifying a pre-existing plurality of probe nucleic acids. In some embodiments, modifying the pre-existing plurality of probe nucleic acids may comprise adding one or more probes. In some embodiments, about one probe, two probes, three probes, four probes, five probes, six probes, seven probes, eight probes, nine probes, ten probes, twenty probes, thirty probes, forty probes, fifty probes, sixty probes, seventy probes, eighty probes, ninety probes, about one hundred probes, or about over one hundred probes may be added. In some embodiments, modifying the pre-existing plurality of probe nucleic acids may comprise removing one or more probes. In some embodiments, about one probe, two probes, three probes, four probes, five probes, six probes, seven probes, eight probes, nine probes, ten probes, twenty probes, thirty probes, forty probes, fifty probes, sixty probes, seventy probes, eighty probes, ninety probes, about one hundred probes, or about over one hundred probes may be removed. In some embodiments, generating may comprise performing a comparison between the one or more biomarkers and a set of previously identified biomarkers. In some embodiments, the one or more biomarkers may comprise tumor-associated alterations. In some embodiments, the previously identified biomarkers may comprise tumor-associated alterations. In some embodiments, the tumor-associated alterations may comprise one or more of: single nucleotide variants (SNVs), insertions or deletions (indels), or rearrangements, or any combination thereof. In some embodiments, the one or more biomarkers may comprise differentially expressed markers or variants. In some embodiments, the previously identified biomarkers may comprise differentially expressed markers or variants. In some embodiments, the previously identified biomarkers may be identified prior to the administration of a treatment regimen to the subject. In some embodiments, the previously identified biomarkers may be identified during the administration of a treatment regimen of the subject. In some embodiments, the previously identified biomarkers may comprise biomarkers that were identified at plurality of different time points.

In some embodiments, the plurality of different time points may comprise at least two time points. In some embodiments, the plurality of different time points may comprise about at least three, four, five, six, seven, eight, nine, ten, eleven, twelve, thirteen, fourteen, fifteen, sixteen, seventeen, eighteen, nineteen, or about twenty time points. In some embodiments, the plurality of different time points may occur during the administration of a treatment regimen of the subject. In some embodiments, the method may comprise detecting the presence or absence of minimal residual disease in the subject at an accuracy of at least about 60%. In some embodiments, the method may comprise detecting the presence or absence of MRD in the subject by an accuracy of about at least about 70%, at least about 75%, at least about 80%, at least about 85%, at least about 90%, at least about 95%, at least about 98%, or at least about 99%. In some embodiments, the method comprises detecting the presence or absence of minimal residual disease in the subject at a negative predictive value of at least about 60%, at least about 70%, at least about 75%, at least about 80%, at least about 85%, at least about 90%, at least about 95%, at least about 98%, or at least about 99%. In some embodiments, the method may further comprise detecting the presence or absence of minimal residual disease in the subject at a sensitivity of at least about 60%, at least about 70%, at least about 75%, at least about 80%, at least about 85%, at least about 90%, at least about 95%, at least about 98%, or at least about 99%. In some embodiments, the method may comprise detecting the presence or absence of minimal residual disease in the subject at a specificity of at least about 60%, at least about 70%, at least about 75%, at least about 80%, at least about 85%, at least about 90%, at least about 95%, at least about 98%, or at least about 99%. In some embodiments, the method may comprise detecting the presence or absence of minimal residual disease in the subject at a positive predictive value of at least about 60%, at least about 70%, at least about 75%, at least about 80%, at least about 85%, at least about 90%, at least about 95%, at least about 98%, or at least about 99%.

In some embodiments, the first biological sample may be one or more of: a cell-free deoxyribonucleic acid (cfDNA) sample, a cell-free ribonucleic acid (cfRNA) sample, a plasma sample, a serum sample, a buffy coat sample, a peripheral blood mononuclear cell (PBMC) sample, a red blood cell sample, a urine sample, a urine cell pellet sample, a saliva sample, tissue biopsy, pleural fluid sample, peritoneal fluid sample, amniotic fluid sample, cerebrospinal fluid sample, lymphatic fluid sample, sweat sample, tear sample, semen sample, or any derivative thereof, or any combination thereof. In some embodiments, the first biological sample may comprise a plasma sample. In some embodiments, the first biological sample may comprise a urine sample. In some embodiments, the first biological sample may comprise a tumor tissue sample. In some embodiments, the second biological sample may comprise a urine sample. In some embodiments, the second biological sample may comprise a urine cell pellet sample. In some embodiments, the second biological sample may comprise a blood sample. In some embodiments, the nucleic acid molecules of the first biological sample may comprise DNA molecules.

In some embodiments, the DNA molecules may comprise cell-free DNA (cfDNA) molecules. In some embodiments, the nucleic acid molecules of the first biological sample may comprise RNA molecules. In some embodiments, the RNA molecules may comprise cell-free RNA (cfRNA) molecules.

In another embodiment, disclosed herein is a method for predicting a presence or an absence of treatment-resistant minimal residual disease (MRD) in a subject. In some embodiments, the method may comprise assaying nucleic acid molecules. In some embodiments, the nucleic acid molecules may be from a first biological sample. In some embodiments, the first biological sample may be obtained or derived from the subject at a first time point. In some embodiments, the method may further comprise detecting a first set of biomarkers. In some embodiments, the first set of biomarkers may be detected from the DNA molecules from the first biological sample. In some embodiments, the biomarkers may be detected based at least in part on the assaying of the DNA molecules. In some embodiments, the method may further comprise assaying nucleic acid molecules from a second biological sample. In some embodiments, the second biological sample may be obtained or derived from the subject at a second time point. In some embodiments, the method may further comprise detecting a second set of biomarkers. In some embodiments, the second set of biomarkers may be generated by the nucleic acid molecules from the second biological sample. In some embodiments, the second set of biomarkers may be generated based at least in part on the assaying. In some embodiments, the method may further comprise generating a plurality of probe nucleic acids. In some embodiments, the probe nucleic acids may be customized for the subject. In some embodiments, the probe nucleic acids may comprise sequences corresponding to at least a subset of the first set of biomarkers. In some embodiments, the probe nucleic acids may comprise sequences corresponding to at least a subset of the second set of biomarkers. In some embodiments, the method may further comprise using the plurality of probe nucleic acids to sequence cell free nucleic acids (cfDNA). In some embodiments, the cfDNA may originate from a third biological sample obtained or derived from the subject. In some embodiments, the third biological sample may be obtained or derived from the subject at a third time point. In some embodiments, the third biological sample may be obtained or derived from the subject. In some embodiments, the third biological sample may be used to detect the presence or absence of the subset of the pooled set of biomarkers. In some embodiments, the method may further comprise processing the subset of the pooled set of biomarkers. In some embodiments, processing the subset of the pooled set of biomarkers may be performed to detect the presence of treatment-resistant minimal residual disease (MRD) in the subject.

In some embodiments, the first or second biological sample may be one or more of: a cell-free deoxyribonucleic acid (cfDNA) sample, a cell-free ribonucleic acid (cfRNA) sample, a plasma sample, a serum sample, a buffy coat sample, a peripheral blood mononuclear cell (PBMC) sample, a red blood cell sample, a urine sample, a urine cell pellet sample, a saliva sample, tissue biopsy, pleural fluid sample, peritoneal fluid sample, amniotic fluid sample, cerebrospinal fluid sample, lymphatic fluid sample, sweat sample, tear sample, semen sample, or any derivative thereof, and any combination thereof. In some embodiments, the first or second biological sample may comprise a plasma sample. In some embodiments, the first or second biological sample may comprise a urine sample. In some embodiments, the first or second biological sample may comprise a tumor tissue sample. In some embodiments, the first or second biological sample may be obtained or derived from the subject. In some embodiments, the first or second biological sample may be obtained or derived from the subject using one or more of an ethylenediaminetetraacetic acid (EDTA) collection tube, a cell-free RNA collection tube, or a cell-free deoxyribonucleic acid (DNA) collection tube, other blood collection tube, or CTC collection tubes, or any combination thereof.

In some embodiments, the third biological sample may be selected from one or more of a cell-free deoxyribonucleic acid (cfDNA) sample, a cell-free ribonucleic acid (cfRNA) sample, a plasma sample, a serum sample, a buffy coat sample, a peripheral blood mononuclear cell (PBMC) sample, a red blood cell sample, a urine sample, a urine cell pellet sample, a saliva sample, tissue biopsy, pleural fluid sample, peritoneal fluid sample, amniotic fluid sample, cerebrospinal fluid sample, lymphatic fluid sample, sweat sample, tear sample, semen sample, or any derivative thereof, or any combination thereof. In some embodiments, the third biological sample may comprise the plasma sample. In some embodiments, the third biological sample may comprise the urine sample. In some embodiments, the third biological sample may comprise a urine cell pellet sample. In some embodiments, the third biological sample may comprise a blood sample. In some embodiments, the third biological sample may be obtained or derived from the subject using one or more of an ethylenediaminetetraacetic acid (EDTA) collection tube, a cell-free RNA collection tube, or a cell-free deoxyribonucleic acid (DNA) collection tube, other blood collection tube, or CTC collection tubes, or any combination thereof.

In some embodiments, the method may further comprise detecting the presence or absence of treatment-resistant minimal residual disease in the subject at an accuracy of at least about 60%, at least about 70%, at least about 75%, at least about 80%, at least about 85%, at least about 90%, at least about 95%, at least about 98%, or at least about 99. In some embodiments, the method may further comprise detecting the presence or absence of treatment-resistant minimal residual disease in the subject at a positive predictive value of at least about 60%, at least about 70%, at least about 75%, at least about 80%, at least about 85%, at least about 90%, at least about 95%, at least about 98%, or at least about 99%. In some embodiments, the method may further comprise detecting the presence or absence of treatment-resistant minimal residual disease in the subject in the subject at a negative predictive value of at least about 60%, at least about 70%, at least about 75%, at least about 80%, at least about 85%, at least about 90%, at least about 95%, at least about 98%, or at least about 99%. In some embodiments, the method may further comprise detecting the presence or absence of treatment-resistant minimal residual disease in the subject at a sensitivity of at least about 60%, at least about 70%, at least about 75%, at least about 80%, at least about 85%, at least about 90%, at least about 95%, at least about 98%, or at least about 99%. In some embodiments, the method may further comprise detecting the presence or absence of treatment-resistant minimal residual disease in the subject at a specificity of at least about 60%, at least about 70%, at least about 75%, at least about 80%, at least about 85%, at least about 90%, at least about 95%, at least about 98%, or at least about 99%.

In some embodiments, the third biological sample may be obtained or derived from the subject. In some embodiments, the third biological sample may be obtained or derived using one or more of an ethylenediaminetetraacetic acid (EDTA) collection tube, a cell-free RNA collection tube, or a cell-free deoxyribonucleic acid (DNA) collection tube, other blood collection tube, and CTC collection tubes. In some embodiments, the nucleic acid molecules of the first or second biological sample may comprise DNA molecules. In some embodiments, the DNA molecules may comprise cell-free DNA (cfDNA) molecules. In some embodiments, the nucleic acid molecules of the first or second biological sample may comprise RNA molecules. In some embodiments, the RNA molecules may comprise cell-free RNA (cfRNA) molecules. In some embodiments, the cell free nucleic acids may comprise cfDNA. In some embodiments, the cell free nucleic acids may comprise cfRNA.

In some embodiments, the method may further comprise subjecting the first biological sample to conditions that are sufficient to isolate the DNA molecules. In some embodiments, the method may further comprise subjecting the first biological sample to conditions that are sufficient to enrich the DNA molecules. In some embodiments, the method may further comprise subjecting the first biological sample to conditions that are sufficient to extract the DNA molecules. In some embodiments, the method may further comprise fractionating the first biological sample of the subject. In some embodiments, fractionating the first biological sample of the subject may be performed to obtain the DNA molecules. In some embodiments, the first biological sample may be a whole blood sample. In some embodiments, at least one of the nucleic acid molecules may be assayed using DNA sequencing. In some embodiments, the assaying using DNA sequencing may produce nucleic acid sequencing reads. In some embodiments, the DNA sequencing may comprise whole exome sequencing. In some embodiments, the method may further comprise filtering at least a subset of the nucleic acid sequencing reads. In some embodiments, filtering at least a subset of the nucleic acid sequencing reads may be based on a quality score.

In some embodiments, the method may further comprise performing error correction on the nucleic acid sequencing reads. In some embodiments, the error correction may be performed using sample barcodes. In some embodiments, the error correction may be performed using molecular barcodes. In some embodiments, the barcodes may be attached to at least one of the DNA molecules. In some embodiments, the barcodes may be attached to two of the DNA molecules, three of the DNA molecules, four of the DNA molecules, five of the DNA molecules, or more than five of the DNA molecules. In some embodiments, the method may further comprise performing single-stranded consensus calling. In some embodiments, the method may further comprise performing double-stranded consensus calling. In some embodiments, the method may further comprise performing both single-strand consensus calling and double-stranded consensus calling. In some embodiments, the consensus calling may be performed on the nucleic acid sequencing reads. In some embodiments, the consensus calling may suppress sequencing errors errors in the nucleic acid sequencing reads. In some embodiments, the consensus calling may suppress PCR errors in the nucleic acid sequencing reads. In some embodiments, the sequencing may be performed at a depth of at least 100×. In some embodiments, the sequencing may be performed at a depth of at least 1,000×. In some embodiments, the sequencing may be performed at a depth of at least 10,000×. In some embodiments, the sequencing may be performed at a depth of at least 100,000×. In some embodiments, the sequencing may comprise sequencing nucleic acids derived from the first biological sample.

In some embodiments, the sequencing may comprise sequencing nucleic acids. In some embodiments, the nucleic acids may be derived from the second biological sample. In some embodiments, the assaying may comprise nucleic acid amplification. In some embodiments, the sequencing may comprise nucleic acid amplification. In some embodiments, the nucleic acid amplification may comprise polymerase chain reaction (PCR) amplification. In some embodiments, the nucleic acid amplification may comprise isothermal amplification.

In some embodiments, the cancer may be one or more of: genitourinary cancer, prostate cancer, bladder cancer, or any combination thereof. In some embodiments, the cancer may comprise the bladder cancer. In some embodiments, the bladder cancer may be a muscle invasive bladder cancer. In some embodiments, the subject may be asymptomatic for the cancer. In some embodiments, the first biological sample may be obtained or derived from the subject. In some embodiments, the first biological sample may be obtained or derived from the subject prior to the subject receiving a therapy for the cancer. In some embodiments, the first biological sample or the second biological sample may be obtained or derived from the subject during a therapy for the cancer. In some embodiments, the second biological sample may be obtained or derived from the subject after receiving a therapy for the cancer. In some embodiments, the therapy may be one or more of: surgical resection, chemotherapy, radiotherapy, immunotherapy, cell therapy, adjuvant therapy, neoadjuvant therapy, androgen deprivation therapy, or any combination thereof.

In some embodiments, the first biological sample may be obtained or derived from the subject. In some embodiments, the first biological sample may be obtained or derived from the subject via a transurethral resection of bladder tumor. In some embodiments, the first biological sample may be obtained or derived from the subject after performing a transurethral resection of bladder tumor. In some embodiments, the method may further comprise identifying a clinical intervention for the subject. In some embodiments, the clinical intervention may be based at least in part on the detected presence or the absence of the cancer. In some embodiments, the clinical intervention may be selected from a plurality of clinical interventions. In some embodiments, the clinical intervention may be one or more of, chemotherapy, radiotherapy, immunotherapy, adjuvant therapy, neoadjuvant therapy, androgen deprivation therapy, or a combination thereof. In some embodiments, the surgical resection may be a transurethral resection of bladder tumor (TURBT). In some embodiments, the surgical resection may be a repeat transurethral resection of bladder tumor. In some embodiments, methods and systems described herein may be utilized for ultra-sensitive detection of mutations, such as KRAS mutations. The KRAS mutations may be detected in tumor DNA such as circulating tumor DNA.

In some embodiments, the method may further comprise administering the clinical intervention to the subject. In some embodiments, the plurality of probes may comprise nucleic acid primers. In some embodiments, the plurality of probes may comprise nucleic acid capture probes.

In some embodiments, the plurality of probes may have sequence complementarity with at least a portion of nucleic acid sequences of the set of biomarkers. In some embodiments, the plurality of probes may comprise at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 different probes. In some embodiments, sequencing may comprise using a fixed plurality of probes. In some embodiments, the probes of the fixed plurality of probes may comprise probes that do not comprise sequences of the subset of the set of biomarkers.

In some embodiments, the method may further comprise determining a likelihood of the determination of the presence or the absence of the cancer in the subject. In some embodiments, the method may further comprise monitoring the presence or the absence of the cancer in the subject. In some embodiments, the monitoring may comprise assessing the presence or the absence of the cancer in the subject. In some embodiments, the monitoring may comprise assessing the presence or absence of the cancer in the subject at each of a plurality of time points.

In some embodiments, a difference in the assessment of the presence or the absence of the cancer in the subject may be quantified. In some embodiments, the difference in the assessment of the presence or the absence of cancer in the subject is assessed among the plurality of time points. In some embodiments, the difference in the assessment of the presence or absence of cancer in the subject is indicative of one or more clinical indications. In some embodiments, the one or more clinical indications may be one or more of: a diagnosis of the cancer, a prognosis of the cancer, or an efficacy or non-efficacy of a course of treatment for treating the cancer of the subject, or any combination thereof. In some embodiments, the prognosis may comprise an expected progression-free survival (PFS). In some embodiments, the prognosis may comprise an overall survival (OS). In some embodiments, the method may further comprise determining, among the set of biomarkers, a mutant allele frequency of a set of somatic mutations.

In some embodiments, the method may further comprise determining a circulating tumor DNA (ctDNA) fraction of the cancer of the subject. In some embodiments, determining the ctDNA fraction may be based at least in part on the set of mutant allele frequencies. In some embodiments, the method may further comprise determining a tumor mutational burden (TMB) of the cancer of the subject.

In some embodiments, the method may further comprise determining an abnormality score of the cancer of the subject. In some embodiments, the abnormality score may be based at least in part on the set of mutant allele frequencies. In some embodiments, the method may further comprise applying a treatment. In some embodiments, applying a treatment may be based on the detection of a minimum residual disease. In some embodiments, applying a treatment may be based on the detection of a treatment-resistant minimum residual disease. In some embodiments, the method may further comprise modifying a treatment. In some embodiments, the treatment may be modified based on the detection of the minimum residual disease. In some embodiments, the treatment may be modified based on the treatment-resistant minimum residual disease.

### Integrated Longitudinal MRD Monitoring

In yet another aspect, disclosed herein are systems and methods for integrated RNA and DNA longitudinal MRD monitoring. The MRD detection may be performed using both DNA and RNA samples. Both DNA and RNA may be analyzed with the same probe panel. In some cases, the probe panel may comprise substantially the same probes in the probe array. In some cases, a proportion of the probe panel may comprise substantially the same probes. The probes remaining substantially the same between utilized in DNA and RNA may be core panels of probes. In some cases, the MRD detection may be performed on both cfDNA and cfRNA.

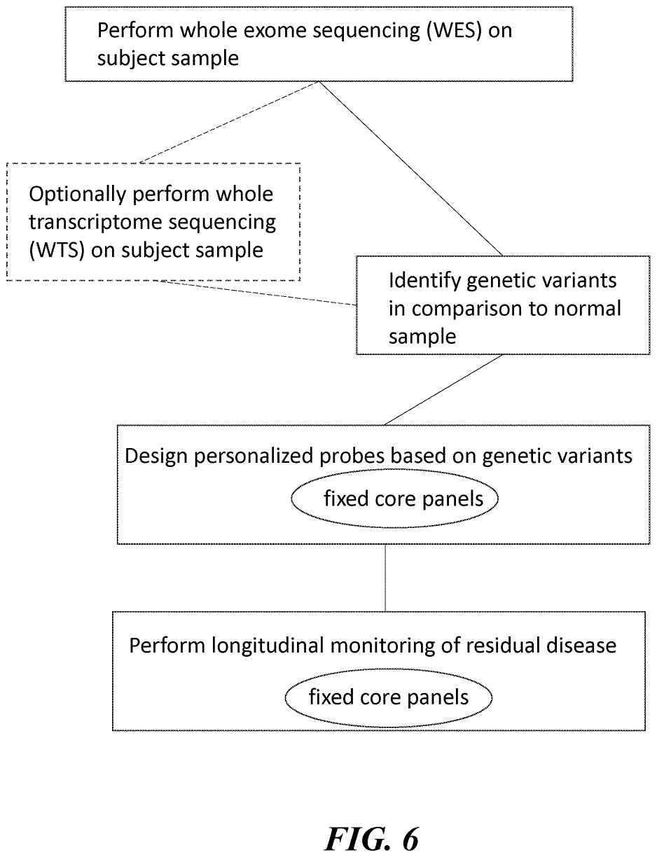

As illustrated in FIG. 6, in some embodiments, integrated RNA and DNA longitudinal MRD monitoring may comprise baseline profiling, personalized mutation panel design, and integrated longitudinal MRD monitoring in DNA and RNA. The longitudinal MRD monitoring may comprise assaying, using the same or a substantially similar probe panel, both DNA and RNA.

In some embodiments, the baseline profiling portion of the systems and methods may comprise performing an assay. The assay may comprise a sequencing assay. The sequencing assay may comprise whole exome sequencing (WES). In some cases, the WES may be performed on one or more tissues, plasma, or urine, or any combination thereof. In some cases, the methods and systems may further comprise optionally performing an assay comprising whole transcriptome sequencing (WTS) as illustrated in FIG. 6.

In some embodiments, the personalized mutation panel design may comprise designing personalized probes. The personalized probes may be designed based on somatic mutations. The personalized probes may be designed based on genetic fusions. In some cases, the personalized probes may comprise a panel of probes. The panel of probes may be a personalized panel. The panel may be a fixed panel. The panel may be a DNA panel. The panel may be an RNA panel. In some cases, the panel may be a core panel, having the same probes for the DNA panel and the RNA panel. At least a portion of the probes of the panel, comprising the core panel, may be the same for DNA and RNA use.

In some embodiments, the longitudinal MRD monitoring may be performed in both DNA and RNA. The MRD monitoring may be performed in parallel for both DNA and RNA. In some cases, at least a portion of the panel may be the same core panel of probes for both DNA and RNA. The core panel may be applied to assays for both cfDNA and cfRNA. The assays using the core panel may be performed on cfDNA and cfRNA samples at one or more time points. The amount of time between a first time point of the core panel assay and a second time point of the core panel assay may be hours, days, weeks, months, or years. In some cases, a report may be generated. The report may comprise an MRD report. In some cases, the report may be an MRD report recommending one or more actions. In some cases, the actions may include recommendations to initiate a treatment, cease a treatment, perform testing, or any combination thereof. In some cases, the report may comprise a determination of whether a DNA mutation is a functional mutation. The DNA mutation may be determined to be a functional mutation or a nonfunctional mutation based at least in part on the RNA core panel assay. The RNA core panel assay may comprise substantially identical probes as the DNA core panel assay.

### Longitudinal MRD Monitoring Integrating Methylation Information, Structural Variants (SVs), and Mutations

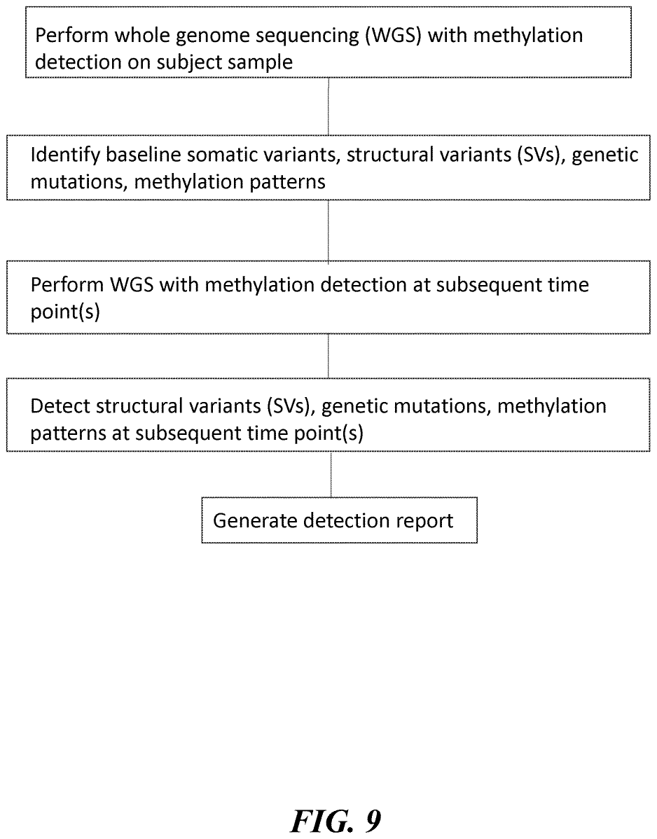

In some examples, as shown in FIG. 9, baseline profiling and MRD monitoring may include determining methylation information of a sample, SVs of a sample, and other mutations of a sample to identify MRD. For example, as illustrated in FIG. 9, baseline profiling may include performing an assay. The assay may comprise whole genome sequencing (WGS). In some cases, the assay may be performed on tissue samples or liquid samples. In some cases, the liquid samples may comprise whole blood or urine from the subject. The blood sample may be processed to extract genomic sequences such as DNA or RNA, or both. In some cases, the sample may comprise cell-free samples. In some cases, methylation of the sample may be detected. In some cases, the WGS may be used to determine epigenetic data. The WGS may be used to determine methylation patterns of genomic data from the sample.

In some embodiments, the WGS may be performed on a control or normal sample. The control or normal sample may originate from the subject. The control or normal sample may originate from a person other than the subject. The WGS may be performed on the control or normal sample to identify one or more baseline somatic variants, such as somatic SVs, for example. In some cases, the one or more baseline somatic variants may comprise SVs and mutations of the normal or control sample.

In some cases, the systems and methods may further comprise performing longitudinal MRD monitoring. The longitudinal MRD monitoring may comprise performing one or more MRD detection assays at one or more time points. In some cases, the MRD assays may comprise WGS assays. In some cases, the WGS assays may comprise methylation WGS assays. The WGS assays may be used to determine methylation data of a sample. The WGS assays may be used to determine a methylation pattern of a sample. In some embodiments, MRD assays may be performed to determine the presence or absence or concentrations of one or more variants. In some cases, the one or more variants may comprise epigenetic information or genomic variants, or both. In some cases, the one or more variants may comprise one or more of: methylation data, structural variants, and mutations. The methylation data may comprise a methylated or unmethylated state of one or more genomic sites, or a methylation pattern of a genomic sequence. In some embodiments, no personalized panel design is required.

In some cases, a report may be generated. The report may comprise an MRD report. In some cases, the report may be an MRD report recommending one or more actions. In some cases, the actions may include recommendations to initiate a treatment, cease a treatment, perform testing, or any combination thereof. In some cases, the report may comprise a determination of whether a DNA mutation is a functional mutation. The report may further comprise a determination of the presence or absence of MRD based at least in part on detection of methylation data, SVs, and mutations. In some cases, the sensitivity and specificity of the MRD may also be contained in the report. In some cases, the sensitivity of the one or more MRD assays such as the WGS may be at least about 50%, 60%, 70%, 80%, 90%, 99%, or more than 99%. In some cases, the specificity of the one or more MRD assays such as the WGS may be at least about 50%, 60%, 70%, 80%, 90%, 99%, or more than 99%. In some cases, the sensitivity of the one or more MRD assays such as WGS including methylation data may be higher as compared to MRD assays such as WGS not including methylation data. In some cases, the specificity of the one or more MRD assays such as WGS including methylation data may be higher as compared to MRD assays such as WGS not including methylation data.

### Computer Control Systems

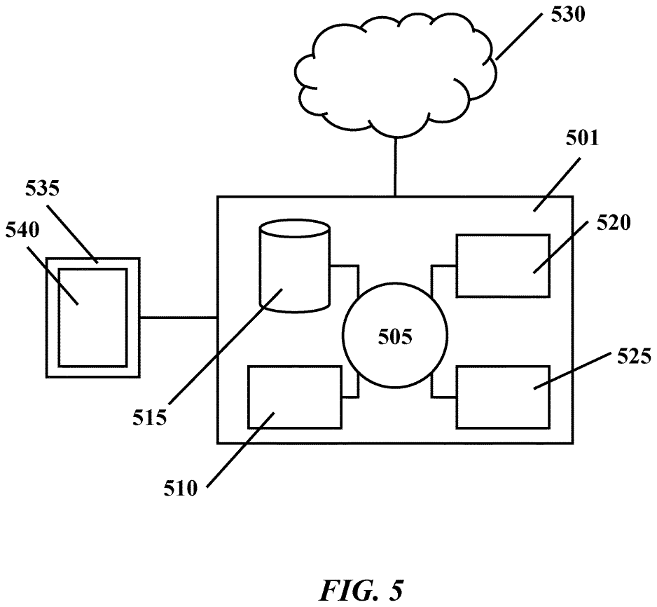

The present disclosure provides computer systems that are programmed to implement methods of the disclosure. FIG. 5 shows a computer system 501 that is programmed or otherwise configured to perform analysis or operations of the methods, for example determine a likelihood of the presence of a cancer based on a set of biomarkers of an individual or run an algorithm. The computer system 501 may regulate various aspects of methods and systems of the present disclosure, such as, for example, perform an algorithm, input training data, analyze sets of biomarker, or output a result for the user as to the presence or absence of cancer. The computer system 501 may be an electronic device of a user or a computer system that is remotely located with respect to the electronic device. The electronic device may be a mobile electronic device.

The computer system 501 includes a central processing unit (CPU, also “processor” and “computer processor” herein) 505, which may be a single core or multi core processor, or a plurality of processors for parallel processing. The computer system 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 may be a data storage unit (or data repository) for storing data. The computer system 501 may be operatively coupled to a computer network (“network”) 530 with the aid of the communication interface 520. The network 530 may be the Internet, an internet and/or extranet, or an intranet and/or extranet that is in communication with the Internet. The network 530 in some cases is a telecommunication and/or data network. The network 530 may include one or more computer servers, which may enable distributed computing, such as cloud computing. The network 530, in some cases with the aid of the computer system 501, may implement a peer-to-peer network, which may enable devices coupled to the computer system 501 to behave as a client or a server.

The CPU 505 may execute a sequence of machine-readable instructions, which may be embodied in a program or software. The instructions may be stored in a memory location, such as the memory 510. The instructions may be directed to the CPU 505, which may subsequently program or otherwise configure the CPU 505 to implement methods of the present disclosure. Examples of operations performed by the CPU 505 may include fetch, decode, execute, and writeback.

The CPU 505 may be part of a circuit, such as an integrated circuit. One or more other components of the system 501 may be included in the circuit. In some cases, the circuit is an application specific integrated circuit (ASIC).

The storage unit 515 may store files, such as drivers, libraries and saved programs. The storage unit 515 may store user data, e.g., user preferences and user programs. The computer system 501 in some cases may include one or more additional data storage units that are external to the computer system 501, such as located on a remote server that is in communication with the computer system 501 through an intranet or the Internet.

The computer system 501 may communicate with one or more remote computer systems through the network 530. For instance, the computer system 501 may communicate with a remote computer system of a user (e.g., a medical professional or patient). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 501 via the network 530.

Methods as described herein may be implemented by way of machine (e.g., computer processor) executable code stored on an electronic storage location of the computer system 501, such as, for example, on the memory 510 or electronic storage unit 515. The machine executable or machine readable code may be provided in the form of software. During use, the code may be executed by the processor 505. In some cases, the code may be retrieved from the storage unit 515 and stored on the memory 510 for ready access by the processor 505. In some situations, the electronic storage unit 515 may be precluded, and machine-executable instructions are stored on memory 510.

The code may be pre-compiled and configured for use with a machine having a processer adapted to execute the code, or may be compiled during runtime. The code may be supplied in a programming language that may be selected to enable the code to execute in a pre-compiled or as-compiled fashion.

Aspects of the systems and methods provided herein, such as the computer system 501, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 501 may include or be in communication with an electronic display 535 that comprises a user interface (UI) 540 for providing, for example, an input of biomarkers or sequencing data, or an visual output relating to a detection, diagnosis, or prognosis. Examples of UI's include, without limitation, a graphical user interface (GUI) and web-based user interface.

Methods and systems of the present disclosure may be implemented by way of one or more algorithms. An algorithm may be implemented by way of software upon execution by the central processing unit 505. The algorithm can, for example, determine a presence or absence of a cancer or cancer parameter based on a set of input sequencing data from a sample derived from a subject.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Method for Longitudinal MRD Monitoring

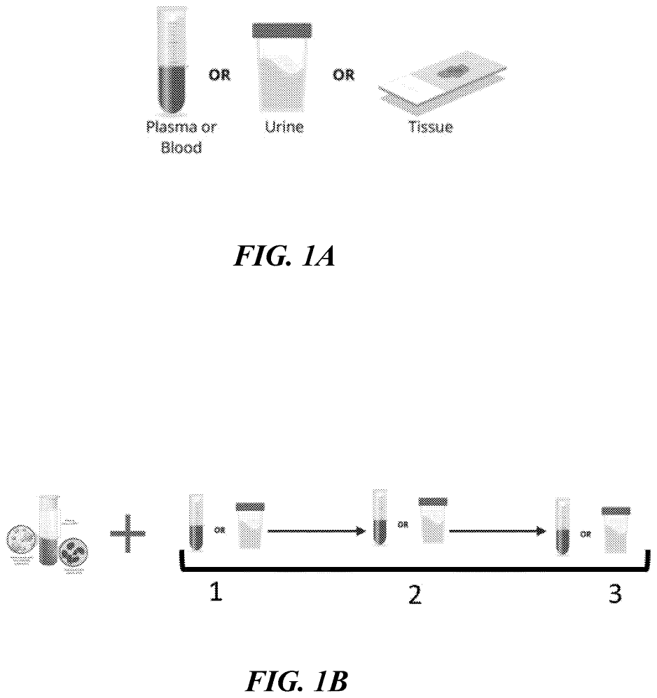

In one non-limiting example, as illustrated in FIG. 1A and FIG. 1B, baseline variant profiling and longitudinal MRD monitoring may be performed. As illustrated in FIG. 1A, for example, baseline variant profiling was performed. A personalized panel was designed without PBMC or tissue. Urine samples were used. As illustrated in FIG. 1A, samples that may be used in baseline variant profiling may comprise plasma or blood, urine, urine cell pellet, or tissue. The panel may be designed without a normal control.

In the non-limiting example illustrated in FIG. 1B, longitudinal MRD monitoring was performed on between 4 and 50 personalized variants. Matched buffy-coat or PBMC samples were sequenced using an MRD panel at 50,000× depth at various points in time as illustrated in FIG. 1B.

### Example 2: Bi-Direction MRD and Resistance Monitoring

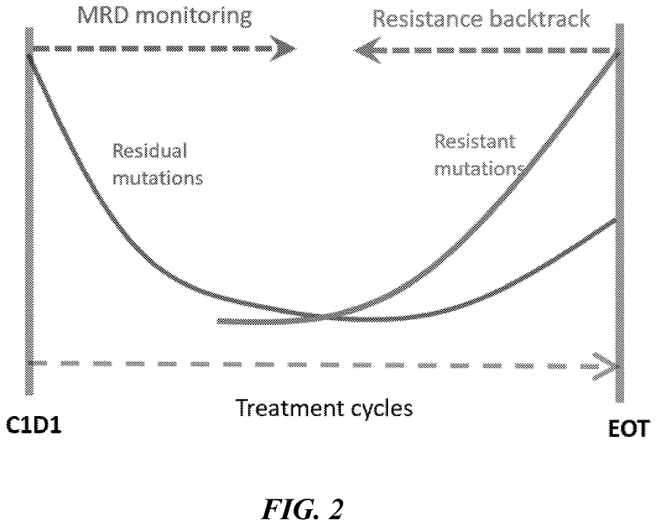

In another non-limiting example, as illustrated in FIG. 2, MRD monitoring may be performed at different time points in one or more treatment cycles in a forward fashion to detect residual mutations. Also as illustrated in FIG. 2, resistance mutation detection may be detected using resistance backtracking from the end of treatment.

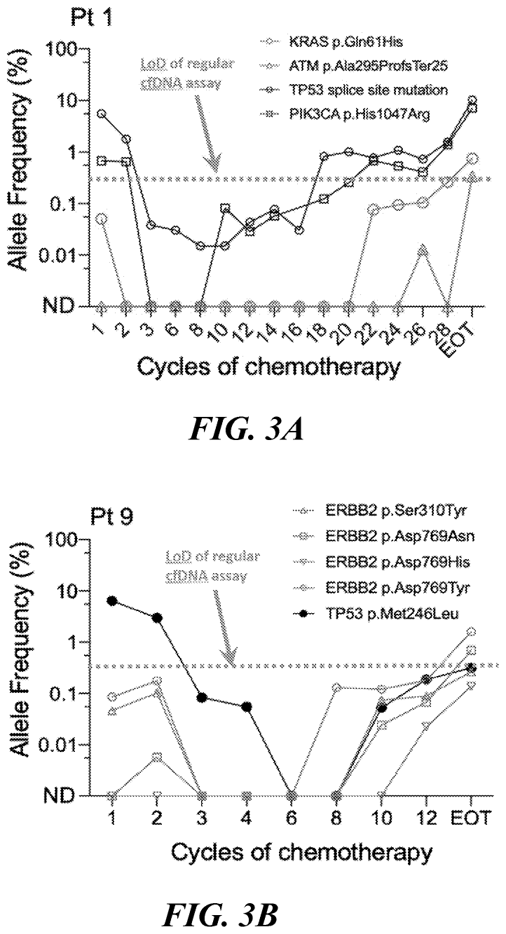

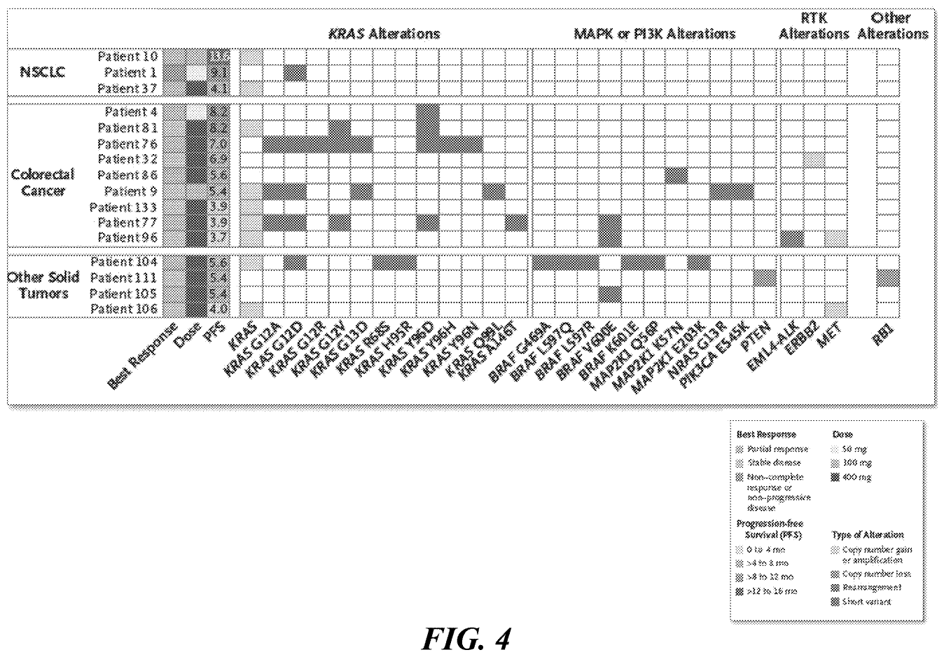

As illustrated in FIG. 3A and FIG. 3B, allele frequency was measured for different genes such as KRAS, ATM, TP53, PIK3CA, and ERBB2 at different time points during treatment, where the time points were chemotherapy cycles. As illustrated in FIG. 4, treatment resistance was mapped using forward and reverse alterations data, such as KRAS gene mutations, MAPK or PI3K alterations, RTK alterations, or other alterations to genes.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

### Example 3: Longitudinal MRD Monitoring Integrating Structural Variants (SVs)

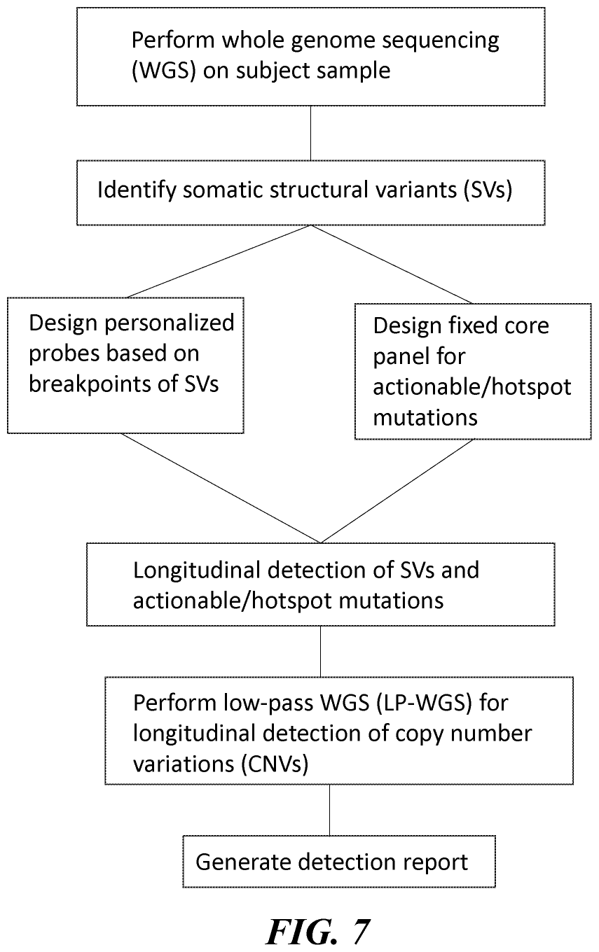

As illustrated in FIG. 7, structural variants (SVs) may be integrated into systems and methods described herein. For example, systems and methods described herein may integrate SVs into baseline profiling, personalized mutation panel design, or longitudinal MRD monitoring, or any combination thereof. In some embodiments, SVs may comprise deletions, insertions, inversions, duplications, translocations, or fusions, or any combination thereof.

For example, as illustrated in FIG. 7, baseline profiling may comprise performing whole genome sequencing (WGS). The WGS can, for example, be performed on tissue, or a liquid such as blood or a derivative thereof. In some cases, the WGS may be performed on a urine cell pellet sample. In some cases, the WGS may be performed on a plasma sample. In some cases, the WGS may be performed on a urine sample. The sample may comprise a cell-free sample. In some cases, the sample may comprise a high tumor fraction sample.

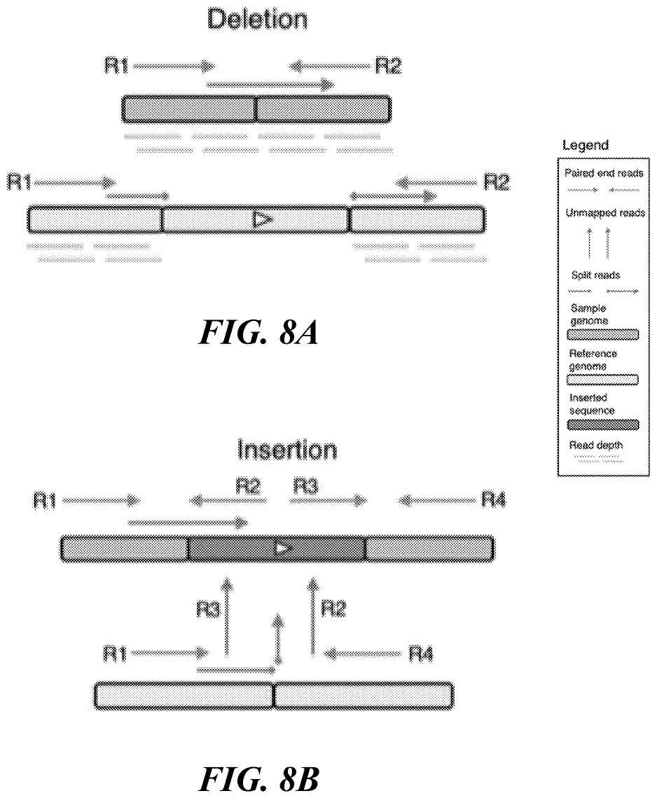

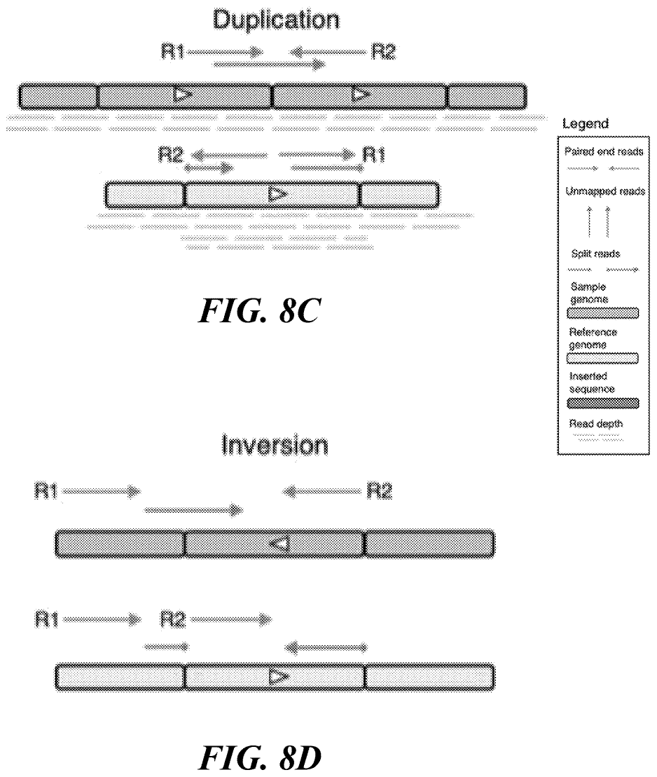

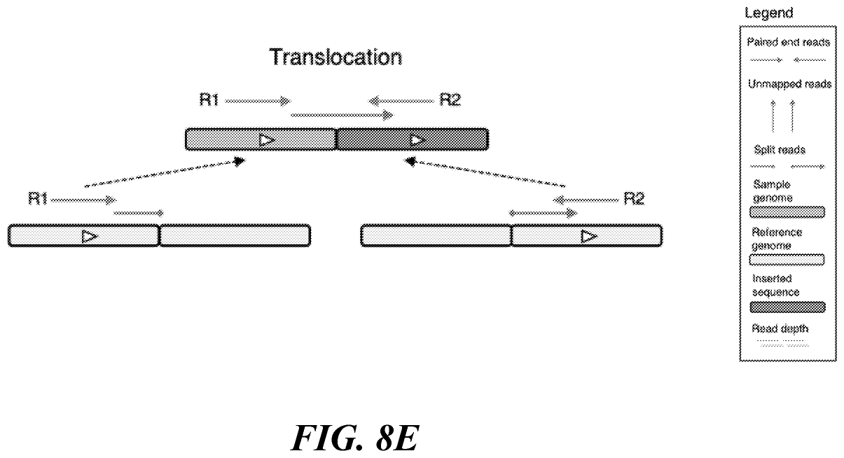

The WGS may be utilized to determine a type of SV. For example, as illustrated in FIGS. 8A-8E, types of SVs may comprise deletion, insertion, inversion, duplication, fusion, and translocation types of SVs. In some cases, A type of somatic SV may be identified in a normal sample. A somatic SV may comprise an SV occurring in a baseline or normal sample of the subject. A somatic SV may comprise an SV occurring in a baseline or normal sample not of the subject. The somatic SV may comprise a control sample, such as a normal sample. For example, SVs may be differentiated by SV types with varying specificity. For example, SVs with high specificity may be selected over other SV types. Determination of specificity of SVs may be performed based at least in part on various SV subtype-specific features. Determination of specificity of SVs may be performed based at least in part on 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more than 50 subtype-specific features of SVs.

As illustrated in FIG. 8A, for example, a deletion SV type may comprise various SV subtype-specific features, such as a varying number of read depths, a direction of identifiable paired end reads, and a direction of identifiable split reads.

As shown in FIG. 8B, an insertion SV type may include, for example, subtype-specific features including various directional unique paired end reads, various directional split reads across an inserted sequence and reference genome, and various unmapped reads at unique locations and of unique lengths.

As shown in FIG. 8C, for example, a duplication SV type may include subtype-specific features including various read depth patterns, various paired end reads of differing directions in the reference and sample genome, and various directional split reads.

As illustrated in FIG. 8D, an inversion SV type may include, for example, subtype-specific features including unique directional paired end reads at unique locations and unique directional split reads at unique locations that may differ between a sample genome and reference genome.

As illustrated in FIG. 8E, for example, a translocation SV type may include, for example, subtype-specific features including unique directional paired end reads at unique locations and unique directional split reads at unique locations that may differ between a sample genome and each of multiple reference genomes.

Next, for example in FIG. 7, the methods and systems may include designing a personalized mutation panel utilizing the SVs. Personalized probes may be designed based on one or more SV breakpoints. Personalized probes may be designed based on a location or position of the one or more SV breakpoints. Personalized probes may be designed based on a relative location or position of the one or more SV breakpoints. In some cases, personalized probes may be designed based at least in part on the distance or number of nucleotides between one or more SV breakpoints. In some cases, personalized probes may be designed based on one or more local genetic or epigenetic characteristics of the SV breakpoints.

In some embodiments, as illustrated in FIG. 7, for example, the personalized mutation panel design may include generating a fixed core panel. The fixed core panel may be targeted to actionable mutations. The fixed core panel may be targeted to hotspot mutations. Actionable mutations may include mutations that may result in a disease or condition. Hotspot mutations may include mutations commonly found at a location in the genome. Hotspot mutations may include a plurality of mutations at a single location or area in the genome, for example.

In some embodiments, as illustrated in FIG. 7, longitudinal MRD monitoring may be performed. Assays for MRD are performed on 30-60 ng of cell-free DNA (cfDNA). Assays for MRD may be performed using a cfDNA sample extracted from 10-20 mL of whole blood or about 20-50 mL of urine. In some cases, SVs may be tracked at one or more time points of MRD monitoring. In some cases, mutations may be determined at one or more time points for MRD monitoring for a subject. For example, SVs or mutations, or both, may be determined at one or more time points days, weeks, months, or years apart. The SVs may comprise somatic SVs.

In some embodiments, longitudinal MRD monitoring may comprise low-pass WGS (LP-WGS). In some cases, the LP-WGS may be utilized to determine genome-wide copy number variations (CNVs). In some cases, CNVs may be determined at one or more time points for MRD monitoring for a subject. In some cases, a report may be generated. The report may comprise an MRD report. In some cases, the report may be an MRD report recommending one or more actions. In some cases, the actions may include recommendations to initiate a treatment, cease a treatment, perform testing, or any combination thereof. In some cases, the report may comprise a determination of whether a DNA mutation is a functional mutation. The report may further comprise a determination of the presence or absence of MRD based at least in part on detection of one or more CNVs, or one or more SVs, or both. The sensitivity of the MRD assays may be included in the report. The sensitivity of the MRD assays may comprise determining the presence of a disease at less than 1 ppm. The sensitivity of the MRD assays may comprise determining the presence of a disease at less than 1 ppm of SVs, CNVs, or other mutations present in the MRD sample.

In some embodiments, the report may comprise a detection by the WGS assay of one or more SVs not previously known. In some cases, the report may comprise a detection by the WGS assay of one or more SV subtypes not previously known. In some cases, the report may comprise a detection by the WGS assay of one or more SVs or SV subtypes unique to the subject.

In some cases, the SVs may be grouped, identified, binned, or detected using one or more subtype features of the SVs. In some cases, the assay reads of genomic DNA or RNA, or both may be aligned using the grouped, identified, binned, or detected SVs. In some cases, the read alignment results of one or more assays, such as WGS assays, may be modified based at least in part on the grouped, identified, binned, or detected SVs. In some cases, the modification of read alignment may remove false alignments. In some cases, the modification of read alignment may add missed alignments.

In some cases, a number, value, proportion, or other amount of SVs may be quantified based at least in part on one or more unique molecular identifiers (UMIs). In some cases, the UMIs may comprise characteristics of the one or more SV subtypes. In some embodiments, split and paired alignments of sequence reads may be combined. In some cases, the combination of split and paired alignments of sequence reads may be utilized for detection of the SVs. In some cases, the combination of split and paired alignments of sequence reads may be utilized for detection of SVs in WGS, LP-WGS, or other MRD assays.

### Example 4: Detection of KRAS Mutations

In some examples, methods and systems described herein may be utilized for ultra-sensitive detection of mutations, such as KRAS mutations. The KRAS mutations may be detected in tumor DNA such as circulating tumor DNA. The detected KRAS mutations may be utilized to determine a presence or absence of a disease.

The Kirsten rat sarcoma viral oncogene homolog (KRAS) gene is mutated in over 90% of pancreatic adenocarcinoma (PDAC). These mutations are predominantly single-base missense variants, 98% of which are found at codon 12 (G12), codon 13 (G13), or codon 61 (Q61). The effects and analysis of pathogenic KRAS G12, G13, or Q61 mutations harbored by circulating tumor DNA (ctDNA) in resectable PDAC prior to treatment initiation using an ultra-sensitive liquid biopsy panel were determined.

Plasma samples for patients with resectable disease at diagnosis were collected prior to chemotherapy initiation. This study cohort included 45 patients (median age 73; 42% male and 58% female; 86.7% Non-Hispanic White, 2.2% Non-Hispanic Black, 8.9% Hispanic White, 2.2% American Indian). At diagnosis, 68.8% had resectable disease (Stage IA-IIA), 15.6% had borderline resectable disease (Stage IIB), and 15.6% had locally advanced disease (Stage III). 38 patients received 3-12 cycles of neoadjuvant chemotherapy with modified FOLFIRINOX and/or Gemcitabine/Abraxane, 27 patients received 1-8 cycles of adjuvant chemotherapy, and 29 patients underwent upfront or interval surgery with Whipple procedure or distal pancreatectomy. Baseline ctDNA was analyzed for KRAS G12/G13/Q61 mutations using the PredicineCARE liquid biopsy assay at 20,000× sequencing depth and the PredicineCARE Ultra assay at more than 100,000× sequencing depth, providing full KRAS gene coverage.

The PredicineCARE assay identified pathogenic KRAS mutations (G12/G13/Q61) in 11 patients. Of these, 8 had co-occurring G12 and Q61 mutations. Presence of any KRAS mutation determined by CARE assay at baseline was not significantly associated with overall survival (OS) (HR 2.96, 95% CI 0.85-10.29), however, detection of a G12 mutation was significantly predictive of worse OS (HR 4.93, 95% CI 1.39-17.44). Co-occurrence of KRAS G12 and Q61 mutations in baseline ctDNA was also predictive of worse OS (HR 3.89, 95% CI 1.12-13.54).

The PredicineCARE Ultra assay identified KRAS mutations in an additional 7 patients who were mutation-negative by the standard CARE assay. Baseline KRAS G12/G13 mutations detected with the Ultra assay were predictive of OS (HR 3.71, 95% CI 1.04-13.28), in addition to demonstrating 100% concordance with CARE assay results for overlapping mutations.

The ultra-sensitive liquid biopsy assay demonstrated robust detection of pertinent KRAS mutations that significantly improves prognostic stratification for PDAC patients.

What is claimed is:

1. A method for identifying a presence or an absence of minimal residual disease (MRD) in a subject, comprising:
(a) assaying nucleic acid molecules from a first biological sample obtained or derived from the subject;
(b) detecting one or more biomarkers from the nucleic acid molecules from the first biological sample based at least in part on the assaying of (a);
(c) generating a plurality of probe nucleic acids that are customized for the subject, wherein the plurality of probe nucleic acids comprise sequences corresponding a set of biomarkers, wherein the set of biomarkers comprise (i) at least a subset of the one or more biomarkers and (ii) one or more previously identified biomarkers;
(d) using the plurality of probe nucleic acids, sequencing cell free nucleic acids from a second biological sample obtained or derived from the subject to detect the presence or absence of a subset of the set of biomarkers;
(e) computer processing the subset of the set of biomarkers to predict the presence of minimal residual disease (MRD) in the subject.

2. The method of claim 1, wherein the generating the plurality of probe nucleic acid comprises modifying a pre-existing plurality of probe nucleic acids by adding or removing one or more probes.

3. The method of claim 1, wherein the generating comprises performing a comparison the one or more biomarkers with a set of previously identified biomarkers.

4. The method of claim 1, wherein the one or more biomarkers or the previously identified biomarkers comprise tumor-associated alterations selected from the group consisting of: single nucleotide variants (SNVs), insertions or deletions (indels), and rearrangements.

5. The method of claim 1, wherein the one or more biomarkers or the previously identified biomarkers comprise differentially expressed markers or variants.

6. The method of claim 1, wherein the previously identified biomarkers were identified prior to the administration of a treatment regimen to the subject.

7. The method of claim 1, wherein the previously identified biomarkers were identified during the administration of a treatment regimen of the subject.

8. The method of claim 1, wherein the previously identified biomarkers comprise biomarkers that were identified at plurality of different time points.

9. The method of claim 1, wherein the plurality of different time points comprises at least two time points during the administration of a treatment regimen of the subject.

10. The method of claim 1, wherein the method comprises detecting the presence or absence of minimal residual disease in the subject at an accuracy of at least about 60%, at least about 70%, at least about 75%, at least about 80%, at least about 85%, at least about 90%, at least about 95%, at least about 98%, or at least about 99%.

11. The method of claim 1, wherein the method comprises detecting the presence or absence of minimal residual disease in the subject in the subject at a negative predictive value of at least about 60%, at least about 70%, at least about 75%, at least about 80%, at least about 85%, at least about 90%, at least about 95%, at least about 98%, or at least about 99%.

12. The method of claim 1, wherein the method comprises detecting the presence or absence of minimal residual disease in the subject at a sensitivity of at least about 60%, at least about 70%, at least about 75%, at least about 80%, at least about 85%, at least about 90%, at least about 95%, at least about 98%, or at least about 99%.

13. The method of claim 1, wherein the method comprises detecting the presence or absence of minimal residual disease in the subject at a specificity of at least about 60%, at least about 70%, at least about 75%, at least about 80%, at least about 85%, at least about 90%, at least about 95%, at least about 98%, or at least about 99%.

14. The method of claim 1, wherein the method comprises detecting the presence or absence of minimal residual disease in the subject at a positive predictive value of at least about 60%, at least about 70%, at least about 75%, at least about 80%, at least about 85%, at least about 90%, at least about 95%, at least about 98%, or at least about 99%.

15. The method of claim 1, wherein the first biological sample is selected from the group consisting of: a cell-free deoxyribonucleic acid (cfDNA) sample, a cell-free ribonucleic acid (cfRNA) sample, a plasma sample, a serum sample, a buffy coat sample, a peripheral blood mononuclear cell (PBMC) sample, a red blood cell sample, a urine sample, a urine cell pellet sample, a saliva sample, tissue biopsy, pleural fluid sample, peritoneal fluid sample, amniotic fluid sample, cerebrospinal fluid sample, lymphatic fluid sample, sweat sample, tear sample, semen sample, or any derivative thereof, and any combination thereof.

16. The method of claim 1, wherein the first biological sample may comprise a plasma sample.

17. The method of claim 1, wherein the first biological sample may comprise a urine sample.

18. The method of claim 1, wherein the first biological sample may comprise a tumor tissue sample.

19. The method of claim 1, wherein the second biological sample may comprise a urine sample.

20. The method of claim 1, wherein the second biological sample may comprise a urine cell pellet sample.
